(19) United States
(12) Patent Application Publication
(10) Pub. No.: US 2024/0165805 A1
(43) Pub. Date: May 23, 2024

(54) COLLISION PREDICTION FOR OPERATING APPARATUS

(71) Applicant: WUHAN UNITED IMAGING HEALTHCARE SURGICAL TECHNOLOGY CO., LTD., Wuhan (CN)

(72) Inventors: Supu YU, Wuhan (CN); Quanquan WANG, Wuhan (CN); Yang ZHANG, Wuhan (CN); Qiang XIE, Shanghai (CN)

(73) Assignee: WUHAN UNITED IMAGING HEALTHCARE SURGICAL TECHNOLOGY CO., LTD., Wuhan (CN)

(21) Appl. No.: 18/420,738

(22) Filed: Jan. 23, 2024

Related U.S. Application Data
(63) Continuation of application No. PCT/CN2022/108644, filed on Jul. 28, 2022.

(30) Foreign Application Priority Data
Jul. 29, 2021 (CN) 202110864436.5

(51) Int. Cl. B25J 9/16; B25J 13/08; B25J 19/02

(52) U.S. Cl. CPC B25J 9/1666; B25J 9/161; B25J 9/163; B25J 9/1676; B25J 13/089; B25J 19/023

(57) ABSTRACT

The present disclosure provides a collision prediction system and method. The method may include obtaining a target model of a target device. The method may also include determining first posture information of the target model and second posture information of an operating apparatus, wherein the target device is set on an end of the operating apparatus. The method may further include generating a collision prediction between the target model and the operating apparatus based on the first posture information and the second posture information, the collision prediction indicating whether there is a space accommodating the target device during an operation of the operating apparatus.

### CROSS-REFERENCE TO RELATED APPLICATIONS

This application is a Continuation of International Application No. PCT/CN2022/108644, filed on Jul. 28, 2022, which claims priority to Chinese Patent Application No. 202110864436.5, filed on Jul. 29, 2021, the contents of which are hereby incorporated by reference

### TECHNICAL FIELD

The present disclosure generally relates to mechanical arms, and more particularly, relates to systems and methods for collision prediction for mechanical arms.

### BACKGROUND

In recent years, tasks in medical diagnosis and/or treatment are often executed using a mechanical arm. In general, a mechanical arm has a higher efficiency and accuracy in executing the tasks. In an operation process, a target device (e.g., a surgical instrument) may be set on an end of the mechanical arm. However, the target device may interfere or collide with the mechanical arm during the operation process since a space for accommodating the target device is often determined according to visual inspection or empirically by a user. Therefore, it may be desirable to provide a system and method for predicting a collision between the target device and the mechanical arm effectively and accurately.

### SUMMARY

According to an aspect of the present disclosure, a system is provided. The system may include at least one storage medium including a set of instructions; and at least one processor configured to communicate with the at least one storage medium, wherein when executing the set of instructions. The at least one processor is configured to direct the system to perform operations including obtaining a target model of a target device; determining first posture information of the target model and second posture information of an operating apparatus, wherein the target device is set on an end of the operating apparatus; and generating a collision prediction between the target model and the operating apparatus based on the first posture information and the second posture information, the collision prediction indicating whether there is a space accommodating the target device during an operation of the operating apparatus.

According to another aspect of the present disclosure, a method implemented on a computing device having a processor and a computer-readable storage device is provided. The method may include obtaining a target model of a target device; determining first posture information of the target model and second posture information of an operating apparatus, wherein the target device is set on an end of the operating apparatus; and generating a collision prediction between the target model and the operating apparatus based on the first posture information and the second posture information, the collision prediction indicating whether there is a space accommodating the target device during an operation of the operating apparatus.

According to a further aspect of the present disclosure, a non-transitory readable medium including at least one set of instructions is provided. When executed by at least one processor of a computing device, the at least one set of instructions may direct the at least one processor to perform a method. The method may include obtaining a target model of a target device; determining first posture information of the target model and second posture information of an operating apparatus, wherein the target device is set on an end of the operating apparatus; and generating a collision prediction between the target model and the operating apparatus based on the first posture information and the second posture information, the collision prediction indicating whether there is a space accommodating the target device during an operation of the operating apparatus.

In some embodiments, the operating apparatus includes a mechanical arm, and the target device is set on an end of the mechanical arm.

In some embodiments, the obtaining a target model of a target device includes: identifying, according to a target identification of the target device, the target model of the target device from a plurality of device models stored in a storage device; and retrieving the target model from the storage device.

In some embodiments, the obtaining a target model of a target device includes: generating the target model of the target device in response to a model generation operation.

In some embodiments, the generating the target model of the target device includes: obtaining three-dimensional information of the target device; and generating the target model of the target device based on the three-dimensional information.

In some embodiments, the three-dimensional information of the target device is acquired by at least one of a structured-light camera, a binocular camera, or a distance measuring device.

In some embodiments, the generating the target model of the target device includes: obtaining critical posture information of the operating apparatus at one or more critical statuses; determining, based on the critical posture information, critical model information of one or more critical models of the target device each of which corresponds to one of the one or more critical statuses of the operating apparatus; and generating the target model of the target device based on the critical model information of the one or more critical models of the target device.

In some embodiments, the critical model information includes dimension information of the one or more critical models.

In some embodiments, the determining, based on the critical posture information, critical model information of one or more critical models of the target device each of which corresponds to one of the one or more critical statuses of the operating apparatus includes: for each of the one or more critical statuses of the operating apparatus, determining, based on critical posture information of the operating apparatus at the critical status, a growth origin and a growth direction of a critical model that corresponds to the critical status; and determining the critical model information of the critical model according to the growth origin and the growth direction.

In some embodiments, the generating the target model of the target device based on the critical model information of the one or more critical models of the target device includes: determining target model information by combining the critical model information of the one or more critical models; and generating the target model of the target device based on the target model information.

According to a further aspect of the present disclosure, a system is provided. The system may include at least one storage medium including a set of instructions; and at least one processor configured to communicate with the at least one storage medium, wherein when executing the set of instructions. The at least one processor is configured to direct the system to perform operations including obtaining critical posture information of an operating apparatus at one or more critical statuses; determining, based on the critical posture information, critical model information of one or more critical models of a target device each of which corresponds to one of the one or more critical statuses of the operating apparatus; generating a target model of the target device based on the critical model information of the one or more critical models of the target device; determining first posture information of the target model and second posture information of the operating apparatus; and generating a collision prediction between the target model and the operating apparatus based on the first posture information and the second posture information, the collision prediction indicating whether there is a space accommodating the target device during an operation of the operating apparatus.

According to a further aspect of the present disclosure, a method implemented on a computing device having a processor and a computer-readable storage device is provided. The method may include obtaining critical posture information of an operating apparatus at one or more critical statuses; determining, based on the critical posture information, critical model information of one or more critical models of a target device each of which corresponds to one of the one or more critical statuses of the operating apparatus; generating a target model of the target device based on the critical model information of the one or more critical models of the target device; determining first posture information of the target model and second posture information of the operating apparatus; and generating a collision prediction between the target model and the operating apparatus based on the first posture information and the second posture information, the collision prediction indicating whether there is a space accommodating the target device during an operation of the operating apparatus.

According to still a further aspect of the present disclosure, a non-transitory readable medium including at least one set of instructions is provided. When executed by at least one processor of a computing device, the at least one set of instructions may direct the at least one processor to perform a method. The method may include obtaining critical posture information of an operating apparatus at one or more critical statuses; determining, based on the critical posture information, critical model information of one or more critical models of a target device each of which corresponds to one of the one or more critical statuses of the operating apparatus; generating a target model of the target device based on the critical model information of the one or more critical models of the target device; determining first posture information of the target model and second posture information of the operating apparatus; and generating a collision prediction between the target model and the operating apparatus based on the first posture information and the second posture information, the collision prediction indicating whether there is a space accommodating the target device during an operation of the operating apparatus.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a block diagram illustrating an exemplary collision prediction system according to some embodiments of the present disclosure;

FIG. 2 is a schematic diagram illustrating exemplary hardware and/or software components of a computing device 200 according to some embodiments of the present disclosure;

FIG. 3 is a schematic diagram illustrating exemplary hardware and/or software components of a mobile device 300 according to some embodiments of the present disclosure;

FIG. 4 is a block diagram illustrating an exemplary processing device according to some embodiments of the present disclosure;

FIG. 5 is a flowchart of an exemplary process for generating a collision prediction between a target model of a target device and an operating apparatus according to some embodiments of the present disclosure;

FIG. 6 is a flowchart of an exemplary process for identifying and retrieving a target model from a storage device according to some embodiments of the present disclosure;

FIG. 7 is a flowchart of an exemplary process for generating a target model of a target device according to some embodiments of the present disclosure;

FIG. 8 is a flowchart of an exemplary process for generating a target model of a target device according to some embodiments of the present disclosure;

FIG. 9A illustrates a critical status of an operating apparatus according to some embodiments of the present disclosure;

FIG. 9B illustrates a critical status of an operating apparatus according to some embodiments of the present disclosure;

FIG. 10 illustrates a first coordinate system associated with an end of an operating apparatus and a second coordinate system associated with a fixing element according to some embodiments of the present disclosure;

FIG. 11 is a flowchart of an exemplary process for determining critical model information of a critical model corresponding to a critical status according to some embodiments of the present disclosure;

FIG. 12A illustrates a critical model of a target device according to some embodiments of the present disclosure;

FIG. 12B illustrates a critical model of a target device according to some embodiments of the present disclosure;

FIG. 13 is a flowchart of an exemplary process for generating a target model of a target device based on critical model information of one or more critical models according to some embodiments of the present disclosure;

FIG. 14 is a flowchart of an exemplary process for generating a target model of a target device according to some embodiments of the present disclosure;

FIG. 15 is a flowchart of an exemplary process for determining target model information of a target model of a target device according to some embodiments of the present disclosure; and

FIG. 16 is a flowchart of an exemplary process for generating a collision prediction between a target model of a target device and an operating apparatus according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

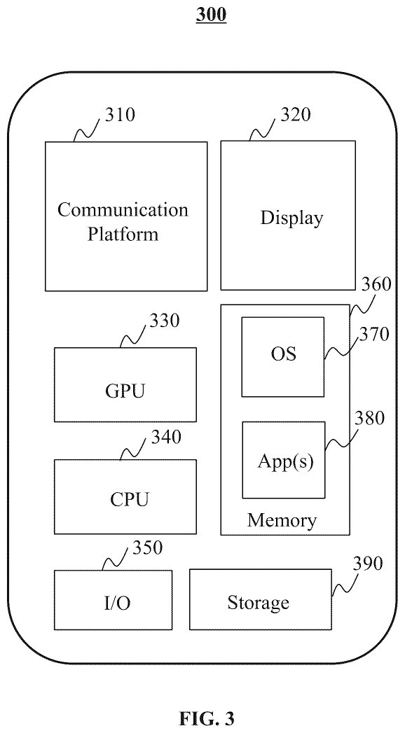

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 3) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

An aspect of the present disclosure relates to a collision prediction system and method. To predict an interference or a collision between a target device (e.g., a surgical instrument) and an operating apparatus (e.g., a mechanical arm), a target model for simulating the target device may be obtained. The target device may be set on an end of the operating apparatus. First posture information of the target model and second posture information of the operating apparatus during an operation of the operating apparatus may be determined. A collision prediction between the target model and the operating apparatus may be generated based on the first posture information and the second posture information. The collision prediction may indicate whether there is a space accommodating the target device. In this way, the collision prediction that indicates whether there is a space accommodating the target device may be more accurate than a conventional way that depends on visual inspection, personal judgment on the spot, and/or experience of a user, thereby saving time and manpower in the medical treatment, reducing cross-user variations, avoiding device collision accidents, and improving the efficiency and/or accuracy in medical diagnosis and/or treatment.

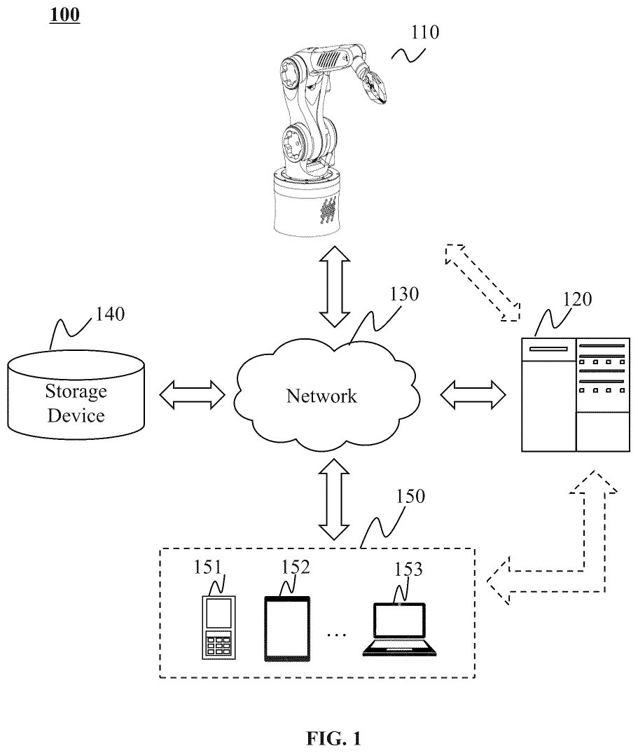

FIG. 1 is a block diagram illustrating an exemplary collision prediction system according to some embodiments of the present disclosure. The collision prediction system 100 may monitor and/or predict an interference or collision between an operating apparatus and a target device set on an end of the operating apparatus during an operation of the operating apparatus. In some embodiments, the collision prediction system 100 may be an intelligent system, which may be applicable in industrial manufacturing, medical imaging and/or treatment, catering services, hairdressing, smart home services, etc. For better understanding the present disclosure, an application scenario of the collision prediction system 100 in a medical treatment is described as an example, which is not intended to limit the scope of the present disclosure.

As shown in FIG. 1, the collision prediction system 100 may include an operating apparatus 110, a processing device 120, a network 130, a storage device 140, and one or more terminal devices 150. In some embodiments, the operating apparatus 110, the processing device 120, the storage device 140, and/or the terminal device 150 may be connected to and/or communicate with each other via a wireless connection (e.g., a wireless connection provided by the network 130), a wired connection (e.g., a wired connection provided by the network 130), or any combination thereof.

The operating apparatus 110 may be a medical treatment apparatus. Exemplary medical treatment apparatuses may include a surgical operating apparatus, a radiation therapy (RT) apparatus, a laser interstitial thermal therapy (LITT) apparatus, or the like, or a combination thereof. The medical treatment apparatus may perform a medical treatment to a subject or a portion thereof using a target device. The target device may include, e.g., a surgical instrument, a radiation source, a treatment probe, etc. The target device may be installed or set on the medical treatment apparatus.

In some embodiments, the subject may include a body, a substance, an object, or the like, or any combination thereof. In some embodiments, the subject may include a specific portion of a body, a specific organ, or specific tissue, etc. For example, the subject may include a lesion (e.g., a tumor) in the brain, the thorax, the stomach, soft tissue, etc., of a patient.

Merely by way of example, the operating apparatus 110 may be a surgical operating apparatus. The surgical operating apparatus may perform a surgical treatment (e.g., selective neurotomy, tumor removal, bone repair, gastric resection, etc.) to a subject using a surgical instrument (e.g., a bistoury, a bone drill, a micro thruster). The surgical instrument may be installed on an end of the surgical operating apparatus. As another example, the operating apparatus 110 may be an LITT apparatus. The LITT apparatus may perform a thermal therapy to a subject using an LITT probe. The LITT probe may be installed on an end of the LITT apparatus.

The processing device 120 may process data and/or information obtained from the operating apparatus 110, the storage device 140, and/or the terminal device 150. For example, the processing device 120 may obtain a target model of the target device, determine first posture information of the target model and second posture information of the operating apparatus 110. Based on the first posture information and the second posture information, the processing device 120 may generate a collision prediction between the target model and the operating apparatus. The collision prediction may indicate whether there is a space sufficient for accommodating the target device during an operation of the operating apparatus. As another example, the processing device 120 may obtain an image of a region including the subject, and guide the target device to arrive a position of the subject accurately based on the image.

In some embodiments, the processing device 120 may be a single server or a server group. The server group may be centralized or distributed. In some embodiments, the processing device 120 may be local to or remote from the operating apparatus 110. For example, the processing device 120 may access information or data from and/or generate instructions for controlling the operating apparatus 110, the storage device 140, and/or the terminal device 150 via the network 130. As another example, the processing device 120 may be directly connected to the operating apparatus 110, the terminal device 150, and/or the storage device 140 to access information and/or data. In some embodiments, the processing device 120 may be implemented on a cloud platform. The cloud platform may include a private cloud, a public cloud, a hybrid cloud, a community cloud, a distributed cloud, an inter-cloud, a multi-cloud, or the like, or any combination thereof.

The network 130 may include any suitable network that can facilitate the exchange of information and/or data for the collision prediction system 100. In some embodiments, one or more components of the collision prediction system 100 (e.g., the operating apparatus 110, the processing device 120, the storage device 140, or the terminal device 150) may communicate information and/or data with one or more other components of the collision prediction system 100 via the network 130. For example, the processing device 120 may obtain parameters of the operating apparatus 110 via the network 130. As another example, the processing device 120 may obtain user instructions from the terminal device 150 via the network 130. The network 130 may include a public network (e.g., the Internet), a private network (e.g., a local area network (LAN), a wide area network (WAN)), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, or the like, or any combination thereof. In some embodiments, the network 130 may include one or more network access points. For example, the network 130 may include wired and/or wireless network access points such as base stations and/or internet exchange points through which one or more components of the collision prediction system 100 may be connected to the network 130 to exchange data and/or information.

The storage device 140 may store data, instructions, and/or any other information. In some embodiments, the storage device 140 may store data obtained from the processing device 120 and/or the terminal device 150. In some embodiments, the storage device 140 may store data and/or instructions that the processing device 120 may execute or use to perform exemplary methods described in the present disclosure. In some embodiments, the storage device 140 may include a mass storage device, a removable storage device, a cloud based storage device, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), a digital versatile disk ROM, etc. In some embodiments, the storage device 140 may be implemented on a cloud platform as described elsewhere in the present disclosure.

In some embodiments, the storage device 140 may be connected to the network 130 to communicate with one or more other components of the collision prediction system 100 (e.g., the processing device 120 or the terminal device 150). One or more components of the collision prediction system 100 may access the data or instructions stored in the storage device 140 via the network 130. In some embodiments, the storage device 140 may be part of the processing device 120.

The terminal device 150 may be connected to and/or communicate with the operating apparatus 110, the processing device 120, and/or the storage device 140. In some embodiments, the terminal device 150 may include a mobile device 151, a tablet computer 152, a laptop computer 153, or the like, or any combination thereof. For example, the mobile device 151 may include a mobile phone, a personal digital assistance (PDA), a laptop, a tablet computer, a desktop, or the like, or any combination thereof. In some embodiments, the terminal device 150 may include an input device, an output device, etc. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to the processing device 120 via, for example, a bus, for further processing. Other types of the input device may include a cursor control device, such as a mouse, a trackball, or cursor direction keys, etc. The output device may include a display, a speaker, a printer, or the like, or any combination thereof. In some embodiments, the terminal device 150 may be part of the processing device 120.

This description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. For example, the processing device 120 may be integrated into the operating apparatus 110. As another example, the collision prediction system 100 may further include a control device configured to control the operating apparatus 110. In some embodiments, the control device may be part of the processing device 120 and/or the terminal device 150. However, those variations and modifications do not depart the scope of the present disclosure.

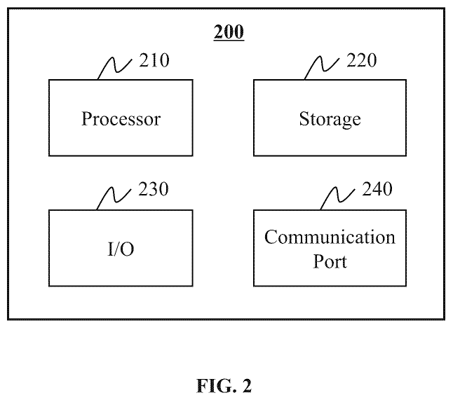

FIG. 2 is a schematic diagram illustrating exemplary hardware and/or software components of a computing device 200 according to some embodiments of the present disclosure. The computing device 200 may be used to implement any component of the collision prediction system 100 as described herein. For example, the processing device 120 and/or the terminal device 150 may be implemented on the computing device 200, respectively, via its hardware, software program, firmware, or a combination thereof. Although only one such computing device is shown, for convenience, the computer functions relating to the collision prediction system 100 as described herein may be implemented in a distributed fashion on a number of similar platforms, to distribute the processing load. As illustrated in FIG. 2, the computing device 200 may include a processor 210, a storage 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process data or information obtained from the operating apparatus 110, the terminal device(s) 150, the storage device 140, and/or any other component of the collision prediction system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors, thus operations and/or method operations that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage 220 may store data/information obtained from the operating apparatus 110, the storage device 140, the terminal device(s) 150, and/or any other component of the collision prediction system 100. In some embodiments, the storage 220 may include a mass storage device, a removable storage device, a volatile read-and-write memory, a read-only memory (ROM), or the like, or any combination thereof. In some embodiments, the storage 220 may store one or more programs and/or instructions to perform exemplary methods described in the present disclosure. For example, the storage 220 may store a program for the processing device 120 to execute for generating an image of a region of interest (ROI) of the subject.

The I/O 230 may input and/or output signals, data, information, etc. In some embodiments, the I/O 230 may enable a user interaction with the processing device 120. In some embodiments, the I/O 230 may include an input device and an output device. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to another component (e.g., the processing device 120) via, for example, a bus, for further processing. Other types of the input device may include a cursor control device, such as a mouse, a trackball, or cursor direction keys, etc. The output device may include a display (e.g., a liquid crystal display (LCD), a light-emitting diode (LED)-based display, a flat panel display, a curved screen, a television device, a cathode ray tube (CRT), a touch screen), a speaker, a printer, or the like, or a combination thereof.

The communication port 240 may be connected to a network (e.g., the network 130) to facilitate data communications. The communication port 240 may establish connections between the processing device 120 and the operating apparatus 110, the terminal device(s) 150, and/or the storage device 140. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee™ link, a mobile network link (e.g., 3G, 4G, 5G), or the like, or a combination thereof. In some embodiments, the communication port 240 may be and/or include a standardized communication port, such as RS232, RS485, etc. In some embodiments, the communication port 240 may be a specially designed communication port. For example, the communication port 240 may be designed in accordance with the digital imaging and communications in medicine (DICOM) protocol.

FIG. 3 is a schematic diagram illustrating exemplary hardware and/or software components of a mobile device 300 according to some embodiments of the present disclosure. In some embodiments, one or more components (e.g., a terminal device 150 and/or the processing device 120) of the collision prediction system 100 may be implemented on the mobile device 300.

As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to the collision prediction system 100. User interactions with the information stream may be achieved via the I/O 350 and provided to the processing device 120 and/or other components of the collision prediction system 100 via the network 130.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

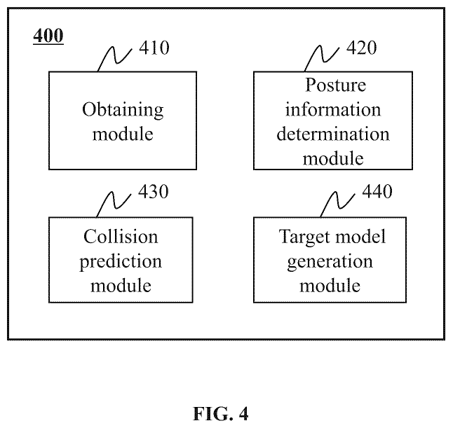

FIG. 4 is a block diagram illustrating an exemplary processing device according to some embodiments of the present disclosure. The processing device 120 may include an obtaining module 410, a posture information determination module 420, a collision prediction module 430, and a target model generation module 440. One or more of the modules of the processing device 120 may be interconnected. The connection(s) may be wireless or wired. At least a portion of the processing device 120 may be implemented on a computing apparatus as illustrated in FIG. 2 or a mobile device as illustrated in FIG. 3.

The obtaining module 410 may obtain data and/or information. In some embodiments, the obtaining module 410 may obtain a target model of a target device. The target model may be a geometric model of the target device. The target model may be used to simulate the target device to predict an interference and collision between the target model and the operating apparatus 110 during a procedure, e.g., a medical treatment. In some embodiments, the obtaining module 410 may identify, according to a target identification of the target device, the target model of the target device from a plurality of device models stored in a storage device, and retrieve the target model from the storage device.

The posture information determination module 420 may determine posture information of the operating apparatus 110 and/or the target device.

The posture information of the operating apparatus 110 at a target posture may also be referred to as second posture information. The posture information of the target model may also be referred to as first posture information. The second posture information of the operating apparatus 110 may include, for example, a position of an end point of each of one or more components of the operating apparatus 110 (e.g., one or more arm sections of the mechanical arm), an angle between each pair of neighboring components of the operating apparatus 110, etc. Merely by way of example, the second posture information of the mechanical arm may include a position of an end point of each of the plurality of arm sections of the mechanical arm, an angle between each pair of neighboring arm sections of the mechanical arm, etc. The first posture information may be determined based on the second posture information. Merely by way of example, the first posture information may be determined by performing a homogeneous transformation on the second posture information.

The collision prediction module 430 may generate a collision prediction between the target model and the operating apparatus.

The collision prediction module 430 may generate the collision prediction by applying the target model of the target device and the model of the operating apparatus 110 according to the first posture information and the second posture information, respectively, into a collision prediction environment. The collision prediction environment may be, for example, a software, a computer program, etc.

The collision prediction may indicate whether there is a space sufficient for accommodating the target device during a procedure implemented as an operation of the operating apparatus 110.

The target model generation module 440 may generate the target model of the target device. In some embodiments, the target model generation module 440 may obtain three-dimensional information of the target device, and generate the target model of the target device based on the three-dimensional information. In some embodiments, the target model generation module 440 may obtain critical posture information of the operating apparatus at one or more critical statuses, determine, based on the critical posture information, critical model information of one or more critical models of the target device each of which corresponds to one of the one or more critical statuses of the operating apparatus, and generate the target model of the target device based on the critical model information of the one or more critical models of the target device.

It should be noted that the above descriptions of the processing device 120 are provided for the purposes of illustration, and not intended to limit the scope of the present disclosure. For persons having ordinary skills in the art, various modifications and changes in the forms and details of the application of the above method and system may occur without departing from the principles of the present disclosure. In some embodiments, the processing device 120 may include one or more other modules. In some embodiments, two or more modules in the processing device 120 may form one module. However, those variations and modifications also fall within the scope of the present disclosure.

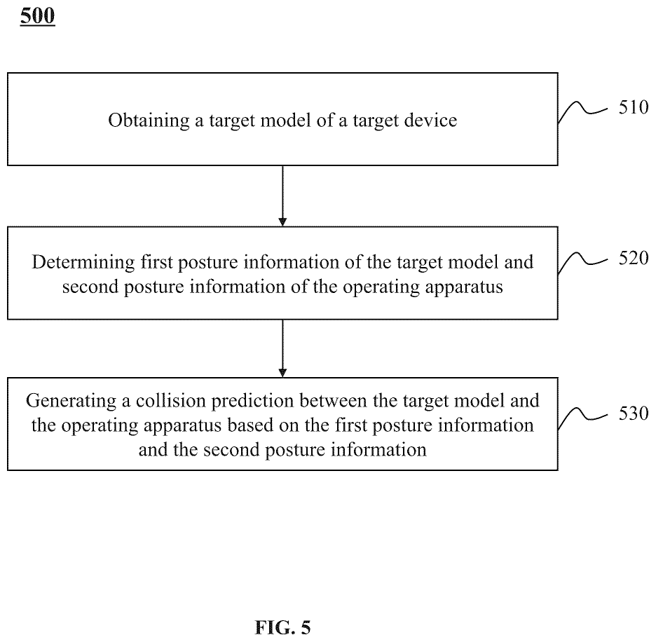

FIG. 5 is a flowchart of an exemplary process for generating a collision prediction between a target model of a target device and an operating apparatus according to some embodiments of the present disclosure. In some embodiments, one or more operations of the process 500 illustrated in FIG. 5 may be implemented in the collision prediction system 100 illustrated in FIG. 1. For example, the process 500 illustrated in FIG. 5 may be stored in the storage device 140 in the form of instructions, and invoked and/or executed by the processing device 120 illustrated in FIG. 1. For illustration purposes, the implement of the process 500 in the processing device 120 is described herein as an example. It should be noted that the process 500 can also be similarly implemented in the terminal device 150.

In 510, the processing device 120 (e.g., the obtaining module 410, the processor 210) may obtain a target model of a target device.

In some embodiments, the target device may include at least one of a plurality of medical devices. The plurality of medical devices may include, e.g., a surgical instrument (e.g., a bone drill, a micro thruster), a treatment probe (e.g., an LITT probe), etc. The target device may be set on an end of the operating apparatus 110.

The operating apparatus 110 may be a medical treatment apparatus (e.g., a surgical operating apparatus, a radiation therapy (RT) apparatus, a laser interstitial thermal therapy (LITT) apparatus, etc.). The medical treatment apparatus may perform a medical treatment to a subject, or a portion thereof, using a target device. In some embodiments, the operating apparatus 110 may include a mechanical arm. Merely for illustration, the mechanical arm may include a base station, a body, an arm, an actuator, and a driving device. The body may be rotatably connected to the base station. One end of the arm may be operably connected to the body and movable relative to the body. The other end of the arm may be operably connected to the target device (e.g., a surgical instrument). The actuator may control a motion of the arm relative to the body. The driving device may be set on the base station and drives the actuator to actuate the arm. The arm may include a plurality of arm sections. A pair of neighboring arm sections may be operably connected by, e.g., a hinge. A desired stability and multiple degrees of freedom (DOF) of the mechanical arm may be realized by such a configuration.

The target model refers to a geometric model of the target device. The target model may be used to simulate the target device to predict an interference and collision between the target model and the operating apparatus 110 during a procedure, e.g., a medical treatment. The target model may have a shape and a size. The shape and/or size of the target model may be determined based on the target device. In some embodiments, the target model may have a shape of, e.g., a cylinder, a cuboid, a cube, a cone, a sphere, etc., or a combination thereof. Merely by way of example, the target model may have a shape of a cylinder. A length of the cylindrical target model may correlate with (e.g., being larger than or equal to) a length of the target device. A diameter of the cylindrical target model may correlate with (e.g., being larger than or equal to) a diameter of a minimum circumscribed cylinder of the target device.

In some embodiments, the target model of the target device may be obtained directly from a storage device (e.g., the storage device 140, the storage 220, the storage 390, a cloud storage, etc.). In some embodiments, the target model of the target device may be generated in response to a model generation operation. Details regarding the obtaining and/or generation of the target model of the target device may be found elsewhere in the present disclosure. See, for example, FIGS. 6-15 and the descriptions thereof.

In 520, the processing device 120 (e.g., the posture information determination module 420, the processor 210) may determine first posture information of the target model and second posture information of the operating apparatus.

After a device model of the target device is obtained, one or more portions of the operating apparatus 110 may be caused to move to achieve a target posture of the operating apparatus 110. For example, at least one arm section of the mechanical arm may be actuated, e.g., by the actuator, to move relative to the base station and/or the body so as to achieve the target posture of the mechanical arm.

The target posture of the operating apparatus 110 may be suitable for a medical treatment. When the operating apparatus 110, holding the target device on an end thereof (e.g., an end surface of an arm section at an end of the mechanical arm), achieves the target posture, the medical treatment may be performed. In some embodiments, target posture of the operating apparatus 110 may be set by a user, according to default settings of the collision prediction system 100, etc.

The processing device 120 may determine posture information of the operating apparatus 110 at the target posture (also referred to as second posture information). The second posture information of the operating apparatus 110 may include, for example, a position of an end point of each of one or more components of the operating apparatus 110 (e.g., one or more arm sections of the mechanical arm), an angle between each pair of neighboring components of the operating apparatus 110, etc. Merely by way of example, the second posture information of the mechanical arm may include a position of an end point of each of the plurality of arm sections of the mechanical arm, an angle between each pair of neighboring arm sections of the mechanical arm, etc. The processing device 120 may also determine posture information of the target model (also referred to as first posture information) when the operating apparatus 110 is at the target posture. The first posture information may be determined based on the second posture information. Merely by way of example, the first posture information may be determined by performing a homogeneous transformation on the second posture information.

In 530, the processing device 120 (e.g., the collision prediction module 430, the processor 210) may generate a collision prediction between the target model and the operating apparatus based on the first posture information and the second posture information.

In some embodiments, a model of the operating apparatus 110 may be generated. The model of the operating apparatus 110 may be generated according to a structure and a size of each of the one or more components of the operating apparatus 110. In some embodiments, the model of the operating apparatus 110 may be generated and stored in a storage device in advance. The collision prediction may be generated by applying the target model of the target device and the model of the operating apparatus 110 according to the first posture information and the second posture information, respectively, into a collision prediction environment. The collision prediction environment may be, for example, a software, a computer program, etc. It should be noted that the collision prediction as set forth in the above embodiment is merely provided for illustration purposes, and not intended to be limiting. Any suitable methods or techniques for generating the collision prediction may be utilized in the present disclosure.

The collision prediction may indicate whether there is a space sufficient for accommodating the target device during a procedure implemented as an operation of the operating apparatus 110. Merely for illustration, if a first result of the collision prediction shows that the target device may interfere or collide with the operating apparatus 110, it may indicate that there is no space sufficient for accommodating the target device during the operation of the operating apparatus 110, and the target device can't be installed on the end of the operating apparatus 110. If a second result of the collision prediction shows that the target device does not interfere or collide with the operating apparatus 110, it may indicate that there is a space sufficient for accommodating the target device during the operation of the operating apparatus 110, the target device can be installed on the end of the operating apparatus 110 to perform the procedure.

According to the embodiments as set forth above, the target device may be simulated using the target model, the operating apparatus 110 may be simulated using a model, as well, so that a collision prediction may be generated based on the target model, the first posture information, and the second posture information. In this way, the collision prediction that indicates whether there is a space sufficient for accommodating the target device may be more accurate than a conventional way that depends on visual inspection or experience of a user, thereby saving time and manpower in the medical treatment, reducing cross-user variations, avoiding device collision accidents, and improving the efficiency and/or accuracy in medical diagnosis and/or treatment.

FIG. 6 is a flowchart of an exemplary process for identifying and retrieving a target model from a storage device according to some embodiments of the present disclosure. In some embodiments, one or more operations of the process 600 illustrated in FIG. 6 may be implemented in the collision prediction system 100 illustrated in FIG. 1. For example, the process 600 illustrated in FIG. 6 may be stored in the storage device 140 in the form of instructions, and invoked and/or executed by the processing device 120 illustrated in FIG. 1. For illustration purposes, the implement of the process 600 in the processing device 120 is described herein as an example. It should be noted that the process 600 can also be similarly implemented in the terminal device 150. In some embodiments, the operation in 510 may be performed according to the process 600. In some embodiments, the operations 610 and 620 may be performed by the obtaining module 410.

In 610, the processing device 120 may identify, according to a target identification of the target device, the target model of the target device from a plurality of device models stored in a storage device.

In some embodiments, a plurality of device models corresponding to various medical devices may be generated and stored in a storage device (e.g., the storage device 140, the storage 220, the storage 390, a cloud storage, etc.) in advance. The various medical devices may include medical devices of different types and/or sizes. The plurality of device models corresponding to the various medical devices may be generated by, for example, the processing device 120, in different ways.

For example, the processing device 120 may obtain a device model generation model. The device model generation model may be a machine learning model. Exemplary machine learning models may include multiple layer perceptron (MLP) model, a gradient boosting decision tree (GBDT) model, an extreme gradient boosting (XGB) model, a logistic regression model, and a factorization machine (FM) model, or the like, or any combination thereof. In some embodiments, the model may be selected from the group consisting of a multiple layer perceptron (MLP) model, a gradient boosting decision tree (GBDT) model, an extreme gradient boosting (XGB) model, a logistic regression model, and a factorization machine (FM) model. The device model generation model may be trained based on multiple sample models corresponding to multiple sample devices. Merely for illustration, the multiple sample models, each of which corresponds to a sample device, may be obtained. Features (e.g., a shape, a size, etc.) of each sample model and a corresponding sample device may be extracted and used to train the device model generation model.

As another example, for each of the various medical devices, the processing device 120 may obtain three-dimensional information of the medical device, and generate a device model corresponding to the medical device based on the three-dimensional information. The three-dimensional information of the medical device may include, for example, dimension information (e.g., a length, a diameter of a minimum circumscribed cylinder of the medical device) of the medical device in the three-dimensional space.

A medical device may have an identification. The identification may relate to a type and/or size of the medical device. The identification may include but not limited to a name, a model number, a code number, etc. An identification of a medical device may be used to distinguish the medical device from other medical devices among the various medical devices.

The target device may have a target identification. The processing device 120 may identify, according to the target identification of the target device, the target model from the plurality of device models.

In 620, the processing device 120 may retrieve the target model from the storage device.

After the target model is identified from the plurality of device models stored in the storage device, the processing device 120 may retrieve the target model from the storage device. The target model may be used to generate the collision prediction between the target model and the operating apparatus 110 as described in 520 and 530 of the process 500 in FIG. 5.

By identifying, according to the target identification of the target device, the target model of the target device from the plurality of device models and retrieving the target model from the storage device, the target model may be obtained directly, thereby saving time and manpower in the medical treatment, and improving the efficiency in medical diagnosis and/or treatment.

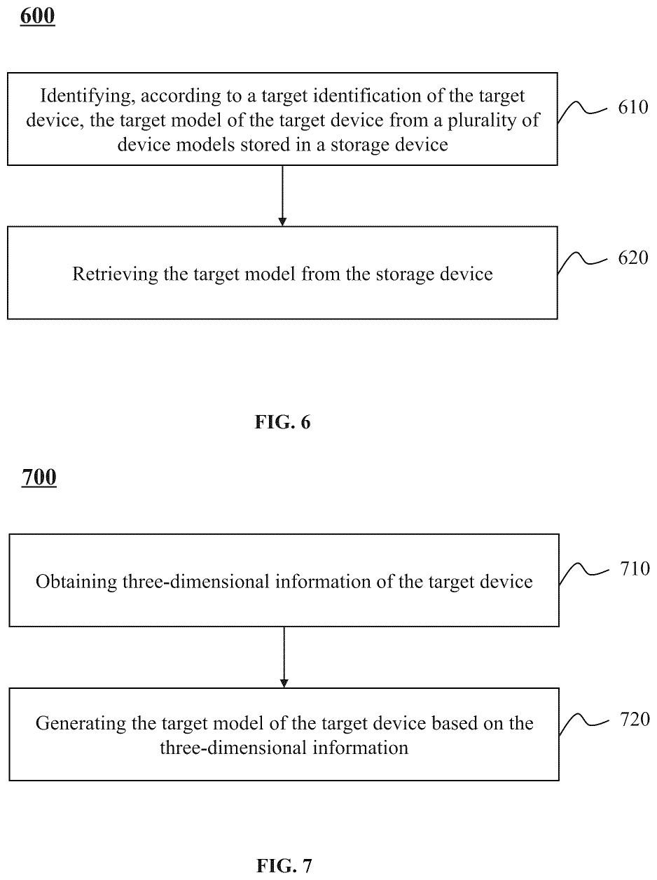

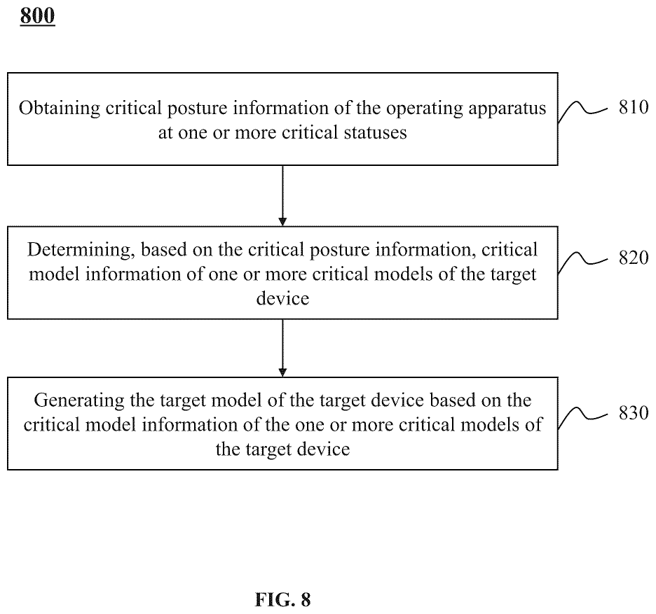

In some embodiments, the target model of the target device may not be generated and stored in the storage device in advance. Instead, the target model may be generated in response to a model generation operation. The model generation operation may include, e.g., a triggering operation (e.g., obtaining three-dimensional information of the target device) for generating the target model, an input operation from a user via, e.g., an interface of the terminal device 150, etc. Details regarding the generation of the target model may be found elsewhere in the present disclosure. See, for example, FIGS. 7 and 8 and the descriptions thereof.

FIG. 7 is a flowchart of an exemplary process for generating a target model of a target device according to some embodiments of the present disclosure. In some embodiments, one or more operations of the process 700 illustrated in FIG. 7 may be implemented in the collision prediction system 100 illustrated in FIG. 1. For example, the process 700 illustrated in FIG. 7 may be stored in the storage device 140 in the form of instructions, and invoked and/or executed by the processing device 120 illustrated in FIG. 1. For illustration purposes, the implement of the process 700 in the processing device 120 is described herein as an example. It should be noted that the process 700 can also be similarly implemented in the terminal device 150. In some embodiments, the operations 710 and 720 may be performed by the model generation module 440.

In 710, the processing device 120 may obtain three-dimensional information of the target device.

The three-dimensional information of the target device may be information relating to a shape, a size (e.g., a length, a diameter of a minimum circumscribed cylinder of the target device), an outline, etc., of the target device in the three-dimensional space. The three-dimensional information of the target device may be acquired by a structured-light camera, a binocular camera, a distance measuring device, etc. The structured-light camera may include a projector and one or more light sensors. The projector may project a striped pattern onto the target device. A surface shape of the target device may distort the stripe pattern. The distortion of the pattern may be recorded by the light sensors, and used to generate the three-dimensional information of the target device. Exemplary distance measuring devices may include a microwave radar, an ultrasound sensor, a laser distance sensor, or the like, or a combination thereof. In some embodiments, in response to the model generation operation, the processing device 120 may obtain the three-dimensional information of the target device.

In 720, the processing device 120 may generate the target model of the target device based on the three-dimensional information.

The processing device 120 may reconstruct the target model of the target device based on the three-dimensional information. Any proper model reconstruction methods and/or algorithms may be used to reconstruct the target model of the target device based on the three-dimensional information, which is not limited in the present disclosure.

FIG. 8 is a flowchart of an exemplary process for generating a target model of a target device according to some embodiments of the present disclosure. In some embodiments, one or more operations of the process 800 illustrated in FIG. 8 may be implemented in the collision prediction system 100 illustrated in FIG. 1. For example, the process 800 illustrated in FIG. 8 may be stored in the storage device 140 in the form of instructions, and invoked and/or executed by the processing device 120 illustrated in FIG. 1. For illustration purposes, the implement of the process 800 in the processing device 120 is described herein as an example. It should be noted that the process 800 can also be similarly implemented in the terminal device 150. In some embodiments, the operations 810 through 830 may be performed by the target model generation module 440.

In 810, the processing device 120 may obtain critical posture information of the operating apparatus at one or more critical statuses.

As used herein, a critical status refers to a status at which the target device is about to collide or interfere with the operating apparatus 110, but barely avoids the collision or interference when the target device is set on an end of the operating apparatus 110. Merely by way of example, under a critical status, the target device held at an end of the operating apparatus 110 is about to collide or interfere with a component of the operating apparatus 110 (e.g., the body of the operating apparatus 110), but barely avoids the collision or interference. In some embodiments, the critical status may be a status at which a surface of a profile of the target device is tangent to a surface of a profile of the operating apparatus 110.

In some embodiments, after the target device is set on the end of the operating apparatus 110, one or more portions of the operating apparatus 110 may be caused to move to traverse various postures of the operating apparatus 110 to identify the one or more critical statuses. For example, at least one arm section of the mechanical arm may be driven, e.g., by the actuator or a user, to move relative to the base station and/or the body so as to traverse various postures of the mechanical arm to identify the one or more critical statuses.

The critical posture information of the operating apparatus 110 refers to posture information of the operating apparatus 110 at one or more critical statuses. In some embodiments, for each of the one or more critical statuses, the critical posture information of a critical status of the operating apparatus 110 may be represented by a posture of a first coordinate system associated with the end of the operating apparatus 110 at the critical status. The first coordinate system may be, e.g., a Cartesian coordinate system including an X_{end} direction, a Y_{end} direction, a Z_{end} direction, and a first origin. The posture of the first coordinate system may characterize the posture of the end of the operating apparatus 110. For example, the Z_{end} direction of the first coordinate system may represent an extension direction of the end of the operating apparatus 110 (e.g., an arm section at the end of the mechanical arm), a plane formed by the X_{end} direction and the Y_{end} direction may represent an end surface of the end of the operating apparatus 110 (e.g., a surface of a flange at the end of the mechanical arm), and the first origin may represent a center point of the end surface of the end of the operating apparatus 110 to which the target device is operably connected. In some embodiments, the posture of the first coordinate system associated with the end of the operating apparatus 110 at each of the one or more critical statuses may be determined according to, e.g., mechanical arm kinematics.

At each of the one or more critical statuses, the processing device 120 may obtain and record critical posture information of the operating apparatus 110. The critical posture information of the operating apparatus 110 may include, for example, a position of an end point or a center point of each of one or more components of the operating apparatus 110, an angle between each pair of neighboring components of the operating apparatus 110, etc., at the critical status.

In 820, the processing device 120 may determine, based on the critical posture information, critical model information of one or more critical models of the target device.

In some embodiments, the target device may be operably connected to the end of the operating apparatus 110 via a fixing element. The fixing element may be used for installing and/or fixing the target device onto the end of the operating apparatus 110. The fixing element may be fixedly connected to the end of the operating apparatus 110. Merely by way of example, the fixing element may include a clamp.

At each of the one or more critical statuses, a posture of a second coordinate system associated with the fixing element may be determined based on the corresponding critical posture information of the operating apparatus 110. The second coordinate system associated with the fixing element may also be a Cartesian coordinate system including an Xₜₒₒₗ direction, a Yₜₒₒₗ direction, a Zₜₒₒₗ direction, and a second origin. The posture of the second coordinate system may characterize the posture of the fixing element and the target device. For example, the Zₜₒₒₗ direction of the second coordinate system may represent an extension direction of the fixing element, the Xₜₒₒₗ direction may represent an installation direction of the target device (e.g., a direction of an installation hole of the fixing element for installing the target device), and the second origin may represent an installation position of the target device (e.g., a center of the installation hole).

As set forth above, the critical posture information of the operating apparatus 110 may be represented by the posture of the first coordinate system associated with the end of the operating apparatus 110. For each of the one or more critical statuses, the posture of the second coordinate system associated with the fixing element may be determined based on the corresponding posture of the first coordinate system associated with the end of the operating apparatus 110. In some embodiments, the posture of the second coordinate system associated with the fixing element may be determined using a transformation matrix. The transformation matrix may transform the first coordinate system into the second coordinate system. Any proper matrix that implements the transformation from the first coordinate system to the second coordinate system may be used as the transformation matrix, which is not limiting in the present disclosure.

After the posture of the second coordinate system associated with the fixing element is determined, the critical model information of a critical model of the target device may be determined.

As used herein, a critical model refers to an intermediate model of the target device at a critical status. Each of the one or more critical models may correspond to one of the one or more critical statuses of the operating apparatus 110. The critical model information of a critical model may be information relating to, for example, a shape, a dimension, an outline or contour, etc., or a combination thereof, of the critical model. For instance, the critical model information of a critical model may include at least information relating to dimensions (also referred to as dimension information) of the critical model.

In some embodiments, a critical model may be in the form of a growth model. The growth model may include a growth origin and a growth direction. For each of the one or more critical statuses, the processing device 120 may determine a growth origin and a growth direction of a critical model that corresponds to the critical status, and determine the critical model information of the critical model according to the growth origin and the growth direction. The growth origin and the growth direction of the critical model may be determined based on the posture of the second coordinate system associated with the fixing element. Details regarding the determination of the critical model information of one or more critical models of the target device may be found elsewhere in the present disclosure. See, for example, FIG. 11 and the descriptions thereof.

In 830, the processing device 120 may generate the target model of the target device based on the critical model information of the one or more critical models of the target device.

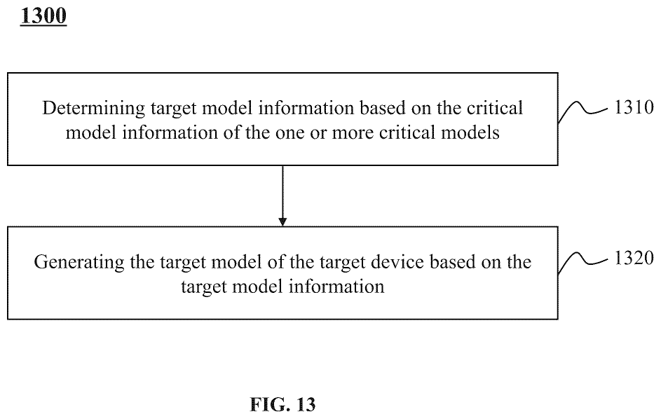

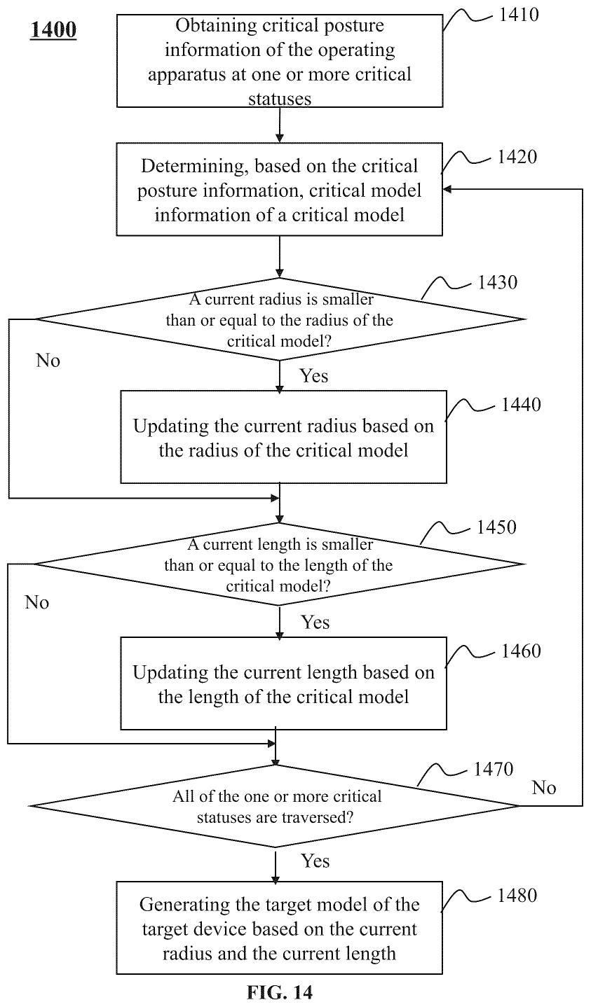

After the critical model information of the one or more critical models of the target device is determined, the processing device 120 may generate the target model of the target device based on the critical model information of the one or more critical models. In some embodiments, the processing device 120 may determine target model information by combining the critical model information of the one or more critical models. The target model information may be information relating to, for example, a shape, a dimension, an outline, etc., of the target model. In some embodiments, the target model information may include information relating to a dimension (also referred to as dimension information) of the target model. The target model of the target device may be generated based on the target model information. Details regarding the generate the target model of the target device based on the critical model information may be found elsewhere in the present disclosure. See, for example, FIGS. 13 and 14 and the descriptions thereof.

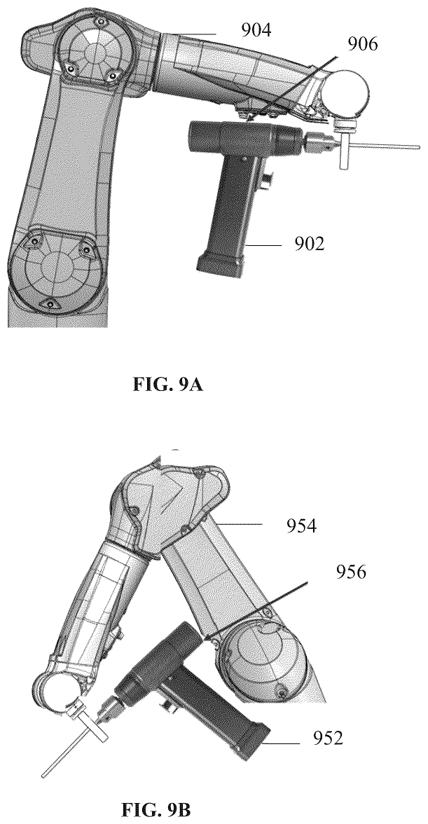

FIG. 9A illustrates a critical status of an operating apparatus according to some embodiments of the present disclosure. As illustrated, a target device 902 may be set on an end of an operating apparatus 904 (e.g., a mechanical arm as shown in the figure). The target device 902 and the operating apparatus 904 may be at a critical status since a surface of a profile of the target device 902 is tangent to a surface of a profile of the operating apparatus 904 at a critical point 906.

FIG. 9B illustrates a critical status of an operating apparatus according to some embodiments of the present disclosure. Similar to FIG. 9A, as illustrated, a target device 952 may be set on an end of an operating apparatus 954 (e.g., a mechanical arm as shown in the figure). The target device 952 and the operating apparatus 954 may be at a critical status since a surface of a profile of the target device 952 is tangent to a surface of a profile of the operating apparatus 954 at a critical point 956.

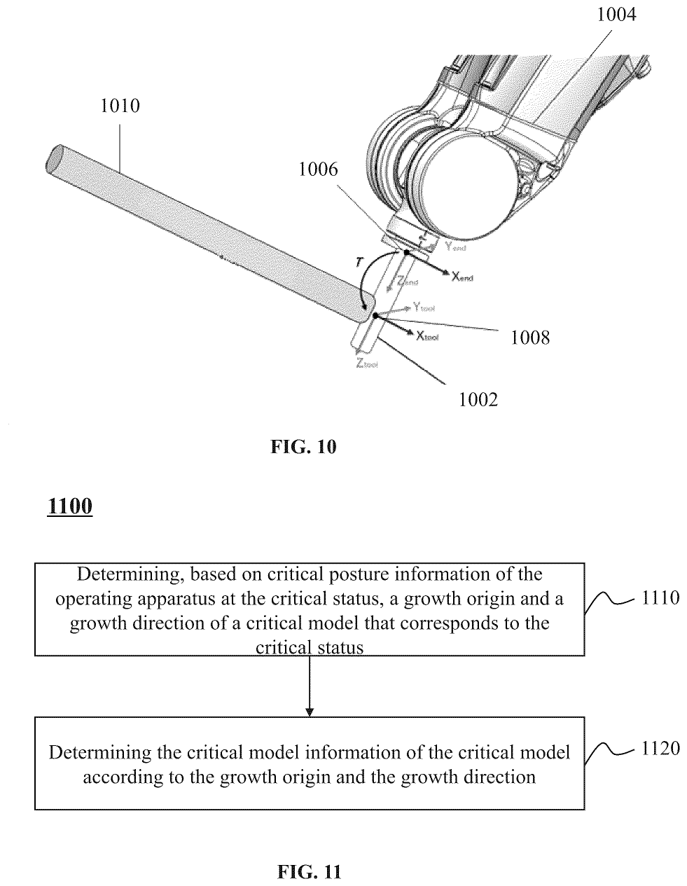

FIG. 10 illustrates a first coordinate system associated with an end of an operating apparatus and a second coordinate system associated with a fixing element according to some embodiments of the present disclosure.

As illustrated in FIG. 10, a fixing element 1002 may be fixedly connected to an end of the operating apparatus 1004. A target device may be operably connected to the end of the operating apparatus 1004 via the fixing element 1002.

The first coordinate system 1006 associated with the end of the operating apparatus 1004 may be defined as a Cartesian coordinate system including an X_{end} direction, a Y_{end} direction, a Z_{end} direction, and a first origin. A posture of the first coordinate system 1006 may characterize a posture of the end of the operating apparatus 1004. For example, the Z_{end} direction of the first coordinate system 1006 may represent an extension direction of the end of the operating apparatus 1004, a plane constituted by the X_{end} direction and the Y_{end} direction may represent an end surface of the end of the operating apparatus 1004 (e.g., a surface of a flange at the end of the mechanical arm), and the first origin may represent a center point of the end surface of the end of the operating apparatus 1004.

The second coordinate system 1008 associated with the fixing element 1002 may also be defined as a Cartesian coordinate system including an Xₜₒₒₗ direction, a Yₜₒₒₗ direction, a Zₜₒₒₗ direction, and a second origin. A posture of the second coordinate system 1008 may characterize a posture of the fixing element 1002 and the target device. For example, the Zₜₒₒₗ direction of the second coordinate system 1008 may represent an extension direction of the fixing element 1002, the Xₜₒₒₗ direction may represent an installation direction of the target device (e.g., a direction of an installation hole of the fixing element 1002 for installing the target device), and the second origin may represent an installation position of the target device (e.g., a center of the installation hole).

For each of the one or more critical status, after the posture of the first coordinate system 1006 associated with the end of the operating apparatus 1004 is obtained, the posture of second coordinate system 1008 associated with the fixing element 1002 may be determined. A critical model 1010 of the target device may be generated based on the posture of second coordinate system 1008 associated with the fixing element 1002.

FIG. 11 is a flowchart of an exemplary process for determining critical model information of a critical model corresponding to a critical status according to some embodiments of the present disclosure. In some embodiments, one or more operations of the process 1100 illustrated in FIG. 11 may be implemented in the collision prediction system 100 illustrated in FIG. 1. For example, the process 1100 illustrated in FIG. 11 may be stored in the storage device 140 in the form of instructions, and invoked and/or executed by the processing device 120 illustrated in FIG. 1. For illustration purposes, the implement of the process 1100 in the processing device 120 is described herein as an example. It should be noted that the process 1100 can also be similarly implemented in the terminal device 150. In some embodiments, the operation 820 of the process 800 may be performed according to the process 1100. In some embodiments, the operations 1110 and 1120 may be performed by the model generation module 440.

In 1110, the processing device 120 may determine, based on critical posture information of the operating apparatus at the critical status, a growth origin and a growth direction of a critical model that corresponds to the critical status.

As set forth above, the critical posture information of the operating apparatus 110 may be represented by the posture of the first coordinate system associated with the end of the operating apparatus 110 at the critical status. The posture of the second coordinate system associated with the fixing element may be determined based on the corresponding posture of the first coordinate system associated with the end of the operating apparatus 110.

After the posture of the second coordinate system associated with the fixing element is determined, the critical model corresponding to the critical status may be determined. In some embodiments, the critical model may be in the form of a growth model. The critical model may include a growth origin and a growth direction. As used herein, the growth origin refers to a start point of the critical model. The growth direction refers to an extension direction of the critical model. Merely for illustration, the growth origin of the critical model may be a center point of an intersection region between an end of the critical model and the fixing element. The growth direction of the critical model may be a direction from the intersection region to another end of the critical mode. The another end of the critical mode may be away from the operating apparatus 110.

Merely for illustration, the second origin of the second coordinate system 1008 as illustrated in FIG. 10 may be determined as the growth origin of the critical model. A direction along a line that connects the second origin of the second coordinate system 1008 and a critical point (e.g., the critical point 906 or 956) or an opposite direction of the Xₜₒₒₗ direction as illustrated in FIG. 10 may be determined as the growth direction of the critical model.

In 1120, the processing device 120 may determine the critical model information of the critical model according to the growth origin and the growth direction.

Merely for illustration, the critical model may be a cylinder. The critical model information of the critical model may include dimension information (e.g., a radius and a length) of the critical model. The dimension information of the critical model may be set to satisfy a critical condition at the critical status. The critical condition at the critical status may be that the critical model of the target device is about to collide or interfere with the operating apparatus 110, but barely avoids the collision or interference (i.e., a surface of a profile of the critical model of the target device may be tangent to a surface of a profile of the operating apparatus 110).

In some embodiments, the radius and the length of the critical model may be initialized as infinity. The radius and/or the length of the critical model may be minimized until critical model of the target device and the operating apparatus 110 satisfy the critical condition at the critical status (i.e., the critical model of the target device is about to collide or interfere with the operating apparatus 110, but barely avoids the collision or interference).

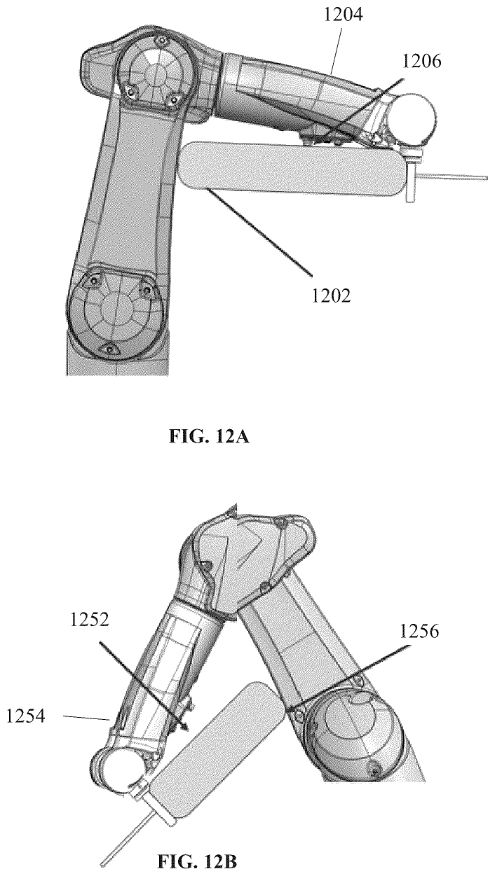

FIG. 12A illustrates a critical model of a target device according to some embodiments of the present disclosure. The critical model 1202 may satisfy a critical condition at the critical status as illustrated in FIG. 9A. The critical condition at the critical status may be that the critical model 1202 is about to collide or interfere with the operating apparatus 1204, but barely avoids the collision or interference (i.e., a surface of a profile of the critical model 1202 may be tangent to a surface of a profile of the operating apparatus 1204 at a critical point 1206 (corresponding to the critical point 906)).

FIG. 12B illustrates a critical model of a target device according to some embodiments of the present disclosure. The critical model 1252 may satisfy a critical condition at the critical status as illustrated in FIG. 9B. The critical condition at the critical status may be that the critical model 1252 the target device is about to collide or interfere with the operating apparatus 1254, but barely avoids the collision or interference (i.e., a surface of a profile of the critical model 1252 may be tangent to a surface of a profile of the operating apparatus 1254 at a critical point 1256 (corresponding to the critical point 956)).

FIG. 13 is a flowchart of an exemplary process for generating a target model of a target device based on the critical model information of the one or more critical models according to some embodiments of the present disclosure. In some embodiments, one or more operations of the process 1300 illustrated in FIG. 13 may be implemented in the collision prediction system 100 illustrated in FIG. 1. For example, the process 1300 illustrated in FIG. 13 may be stored in the storage device 140 in the form of instructions, and invoked and/or executed by the processing device 120 illustrated in FIG. 1. For illustration purposes, the implement of the process 1300 in the processing device 120 is described herein as an example. It should be noted that the process 1300 can also be similarly implemented in the terminal device 150. In some embodiments, the operation 830 of the process 800 may be performed according to the process 1300. In some embodiments, the operations 1310 and 1320 may be performed by the model generation module 440.

In 1310, the processing device 120 may determine target model information based on the critical model information of the one or more critical models.

In some embodiments, the processing device 120 may determine the target model information by combining the critical model information of the one or more critical models. The combination of the critical model information of the one or more critical models may be realized by combining dimension information (e.g., diameters of minimum circumscribed cylinders, lengths) of the one or more critical models. In some embodiments, the combined critical model information may be designated as the target model information of the target model.

Merely for illustration, the one or more critical models may include one or more cylinders. The critical model information of each of the one or more critical models may include dimension information (e.g., a radius and a length) of the critical model. The processing device 120 may identify a minimum radius and a minimum length from radius(es) and length(s) of the one or more critical models, respectively. The target model information may be determined based on the minimum radius and the minimum diameter. In some embodiments, the minimum radius may be designated as a radius of the target model, and the minimum length may be designated as a length of the target model.

In 1320, the processing device 120 may generate the target model of the target device based on the target model information.

After the target model information is determined, the target model of the target device may be reconstructed based on the target model information.

FIG. 14 is a flowchart of an exemplary process for generating a target model of a target device according to some embodiments of the present disclosure. In some embodiments, one or more operations of the process 1400 illustrated in FIG. 14 may be implemented in the collision prediction system 100 illustrated in FIG. 1. For example, the process 1400 illustrated in FIG. 14 may be stored in the storage device 140 in the form of instructions, and invoked and/or executed by the processing device 120 illustrated in FIG. 1. For illustration purposes, the implement of the process 1400 in the processing device 120 is described herein as an example. It should be noted that the process 1400 can also be similarly implemented in the terminal device 150. In some embodiments, the operations 1410 through 1480 may be performed by the model generation module 440.

In 1410, the processing device 120 may obtain critical posture information of the operating apparatus at one or more critical statuses. In some embodiments, the operation 1410 may be similar to or the same as the operation 810 of the process 800 in FIG. 8, which is not repeated here.

In 1420, the processing device 120 may determine, based on the critical posture information, critical model information of a critical model. The critical model information of the critical model may include, for example, a radius and a length of the critical model. In some embodiments, the operation 1420 may be similar to or the same as the operation 820 of the process 800 in FIG. 8 or the operations 1110 and 1120 of the process 1100 in FIG. 11, which is not repeated here.

In 1430, the processing device 120 may determine whether a current radius is smaller than or equal to the radius of the critical model.

An initial value of the current radius may be set as infinity. If it is determined that the current radius is smaller than or equal to the radius of the critical model, the process may proceed to 1450. If it is determined that the current radius is larger than the radius of the critical model, the process may proceed to 1440.

In 1440, the processing device 120 may update the current radius based on the radius of the critical model.

If it is determined that the current radius is larger than the radius of the critical model, the current radius may be replaced with the radius of the critical model.

In 1450, the processing device 120 may determine whether a current length is smaller than or equal to the length of the critical model.

An initial value of the current length may be set as infinity. If it is determined that the current length is smaller than or equal to the length of the critical model, the process may proceed to 1470. If it is determined that the current length is larger than the length of the critical model, the process may proceed to 1460.

In 1460, the processing device 120 may update the current length based on the length of the critical model.

If it is determined that the current length is larger than the length of the critical model, the current length may be replaced with the length of the critical model.

In 1470, the processing device 120 may determine whether all of the one or more critical statuses are traversed.

If it is determined that all of the one or more critical statuses are traversed, the process proceed to 1480. If it is determined that at least a portion of the one or more critical statuses are not traversed, the process proceed to 1420 and the processing device 120 may determine, based on the critical posture information, critical model information of a next critical model.

In 1480, the processing device 120 may generate the target model of the target device based on the current radius and the current length.

The processing device 120 may determine the current radius and the current length as the radius and the length of the target model. The target model of the target device may be generated based on the radius and the length of the target model.

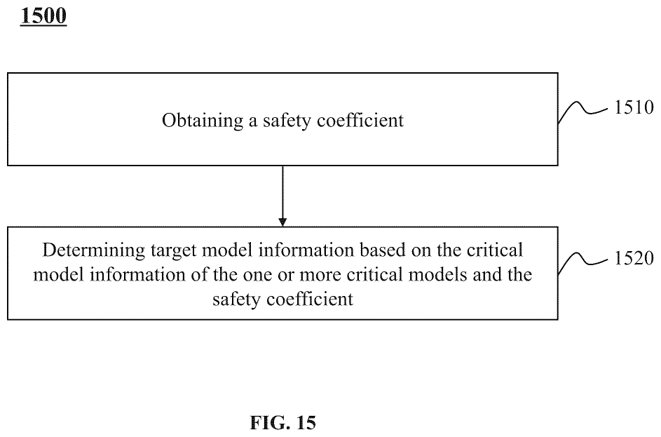

FIG. 15 is a flowchart of an exemplary process for determining target model information of a target model of a target device according to some embodiments of the present disclosure. In some embodiments, one or more operations of the process 1500 illustrated in FIG. 15 may be implemented in the collision prediction system 100 illustrated in FIG. 1. For example, the process 1500 illustrated in FIG. 15 may be stored in the storage device 140 in the form of instructions, and invoked and/or executed by the processing device 120 illustrated in FIG. 1. For illustration purposes, the implement of the process 1500 in the processing device 120 is described herein as an example. It should be noted that the process 1500 can also be similarly implemented in the terminal device 150. In some embodiments, the operation 1310 of the process 1300 in FIG. 13 may be performed according to the process 1500. In some embodiments, the operations 1510 and 1520 may be performed by the model generation module 440.

In 1510, the processing device 120 may obtain a safety coefficient.

The safety coefficient may be a value larger than 1. For example, the safety coefficient may be 1.1, 1.2, 1.3, 1.4, 1.5, 1.6, 1.7, 1.8, 1.9, 2.0, 3.0, etc. The safety coefficient may be set by a user, according to default settings of the collision prediction system 100, etc. In some embodiments, the safety coefficient may be an empirical value or a value determined according to experiments. In some specific scenarios, it is needed to reserve more space for accommodating the target device during an operation of the operating apparatus 110. By setting the safety coefficient, a size of the target model may be larger than an actual size of the target device, thereby reserving more space for accommodating the target device. Merely for illustration, an actual length of the target device may be 30 centimeters, more space may be reserved for accommodating the target device in case of an accident. Therefore, a safety coefficient 1.2 may be provided. By setting the safety coefficient, a length of the target model of the target device may be 36 centimeters, thereby reserving more space for accommodating the target device.

In some embodiments, the safety coefficient may be set by a user through an input operation. For example, the user may input a value into the processing device 120 via an interface of the terminal device 150 or select a value from multiple values of the safety coefficient displayed on the interface of the terminal device 150. The input value or the selected value may be designated as the safety coefficient.

In 1520, the processing device 120 may determine target model information based on the critical model information of the one or more critical models and the safety coefficient.

In some embodiments, the processing device 120 may combine the critical model information of the one or more critical models. The combination of the critical model information of the one or more critical models may be implemented by combining dimension information (e.g., diameters of minimum circumscribed cylinders, lengths) of the one or more critical models. For example, the combination of the critical model information of the one or more critical models may be implemented by identifying a minimum radius and a minimum length from radius(es) and length(s) of the one or more critical models, respectively.

In some embodiments, the processing device 120 may determine target model information by multiplying the combined critical model information with the safety coefficient. Merely for illustration purposes, the one or more critical models may include one or more cylinders. The processing device 120 may identify a minimum radius and a minimum length from radius(es) and length(s) of the one or more critical models, respectively. The target model information may be determined based on the minimum radius, the minimum diameter, and the safety coefficient. In some embodiments, a product of the minimum radius and the safety coefficient may be determined as a radius of the target model, and a product of the minimum length and the safety coefficient may be determined as a length of the target model.

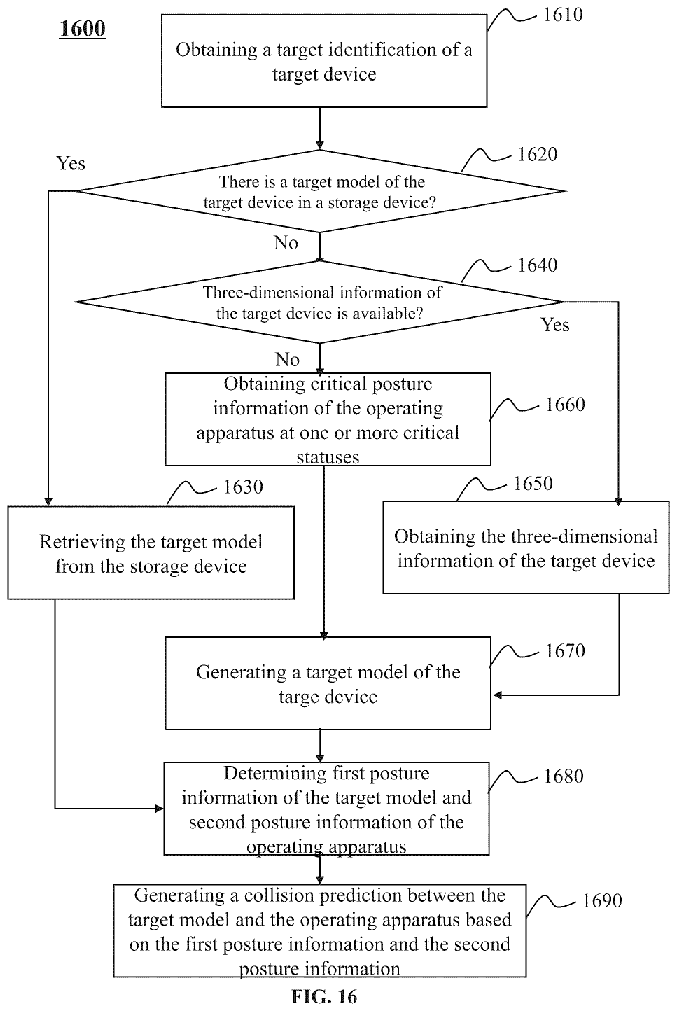

FIG. 16 is a flowchart of an exemplary process for generating a collision prediction between a target model of a target device and an operating apparatus according to some embodiments of the present disclosure. In some embodiments, one or more operations of the process 1600 illustrated in FIG. 16 may be implemented in the collision prediction system 100 illustrated in FIG. 1. For example, the process 1600 illustrated in FIG. 16 may be stored in the storage device 140 in the form of instructions, and invoked and/or executed by the processing device 120 illustrated in FIG. 1. For illustration purposes, the implement of the process 1600 in the processing device 120 is described herein as an example. It should be noted that the process 1600 can also be similarly implemented in the terminal device 150.

In 1610, the processing device 120 may obtain a target identification of a target device.

The target identification may include but not limited to a name, a model number, a code number, etc., of the target device. The target identification may be used to distinguish the target device from other medical devices.

In 1620, the processing device 120 may determine whether there is a target model of the target device in a storage device.

If there is a target model of the target device in the storage device, the process may proceed to 1630. If there is no target model of the target device in the storage device, the process may proceed to 1640.

In 1630, the processing device 120 may retrieve the target model from the storage device.

In some embodiments, the operation 1630 may be similar to or the same as the operation 620 of the process 600 as illustrated in FIG. 6, which is not repeated here.

In 1640, the processing device 120 may determine whether three-dimensional information of the target device is available.

If the three-dimensional information of the target device is available, the process may proceed to 1650. If the three-dimensional information of the target device is unavailable, the process may proceed to 1660.

In 1650, the processing device 120 may obtain the three-dimensional information of the target device.

In some embodiments, the operation 1650 may be similar to or the same as the operation 710 of the process 700 as illustrated in FIG. 7, which is not repeated here.

In 1660, the processing device 120 may obtain critical posture information of the operating apparatus at one or more critical statuses.

In some embodiments, the operation 1660 may be similar to or the same as the operation 810 of the process 800 as illustrated in FIG. 8, which is not repeated here.

In 1670, the processing device 120 may generate a target model of the targe device.

In some embodiments, the operation 1670 may be similar to or the same as the operation 720 of the process 700 as illustrated in FIG. 7 and operations 820 and 830 of the process 800 as illustrated in FIG. 8, which is not repeated here.

In 1680, the processing device 120 may determine first posture information of the target model and second posture information of the operating apparatus.

In some embodiments, the operation 1680 may be similar to or the same as the operation 520 of the process 500 as illustrated in FIG. 5, which is not repeated here.

In 1690, the processing device 120 may generate a collision prediction between the target model and the operating apparatus based on the first posture information and the second posture information.

In some embodiments, the operation 1690 may be similar to or the same as the operation 530 of the process 500 as illustrated in FIG. 5, which is not repeated here.

Merely by way of example, the target device may be a micro thruster. A target identification of the micro thruster may be obtained, and a target model of the micro thruster may be identified according to the target identification. The target model of the micro thruster may be retrieved from a storage device directly. Then first posture information of the target model of the micro thruster and second posture information of the operating apparatus may be determined. A collision prediction between the target model of the micro thruster and the operating apparatus may be generated based on the first posture information and the second posture information. The collision prediction may indicate whether there is a space accommodating the target device during an operation of the operating apparatus.

As another example, the target device may be a bone drill. There is no target model of the bone drill pre-stored in a storage device. In the meanwhile, three-dimensional information of the bone drill is unavailable. In this case, the bone drill may be set on the end of the operating apparatus 110, and one or more portions of the operating apparatus 110 may be controlled to move to traverse various postures of the operating apparatus 110 to identify one or more critical statuses.

For example, three critical statuses may be identified. At a first critical status, critical posture information of the operating apparatus 110 at the first critical status may be obtained, and a first critical model may be reconstructed. The first critical model may have a shape of a cylinder. A radius of the first critical model may be 64 millimeters (mm), and a length of the first critical model may be infinity. In some embodiments, the length of the first critical model may be set to 400 mm. At a second critical status, critical posture information of the operating apparatus 110 at the second critical status may be obtained, and a second critical model may be reconstructed. The second critical model may have a shape of a cylinder. A radius of the second critical model may be 73 mm, and a length of the second critical model may be 200 mm. At a third critical status, critical posture information of the operating apparatus 110 at the third critical status may be obtained, and a third critical model may be reconstructed. The third critical model may have a shape of a cylinder. A radius of the second critical model may be larger than 73 mm, and a length of the second critical model may be between 200 mm and 400 mm.

Target model information of the bone drill may be determined based on the critical model information of the one or more critical models and a safety coefficient. For example, the safety coefficient may be 1.1. A radius of the target model of the bone drill may be 70.4 mm, which is a product of 64 mm and 1.1. A length of the target model of the bone drill may be 220 mm, which is a product of 200 mm and 1.1. The target model of the bone drill may be generated. Then first posture information of the target model of the bone drill and second posture information of the operating apparatus may be determined. A collision prediction between the target model of the bone drill and the operating apparatus may be generated based on the first posture information and the second posture information. The collision prediction may indicate whether there is a space accommodating the target device during an operation of the operating apparatus. By setting the safety coefficient, a size of the target model may be larger than an actual size of the bone drill, thereby reserving more space for accommodating the bone drill.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by the present disclosure, and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system, comprising:
at least one storage medium including a set of instructions; and
at least one processor configured to communicate with the at least one storage medium, wherein when executing the set of instructions, the at least one processor is configured to direct the system to perform operations including:
obtaining a target model of a target device;
determining first posture information of the target model and second posture information of an operating apparatus, wherein the target device is set on an end of the operating apparatus; and
generating a collision prediction between the target model and the operating apparatus based on the first posture information and the second posture information, the collision prediction indicating whether there is a space accommodating the target device during an operation of the operating apparatus.

2. The system of claim 1, wherein the operating apparatus includes a mechanical arm, and the target device is set on an end of the mechanical arm.

3. The system of claim 1, wherein the obtaining a target model of a target device includes:
identifying, according to a target identification of the target device, the target model of the target device from a plurality of device models stored in a storage device; and
retrieving the target model from the storage device.

4. The system of claim 1, wherein the obtaining a target model of a target device includes:
generating the target model of the target device in response to a model generation operation.

5. The system of claim 4, wherein the generating the target model of the target device includes:
obtaining three-dimensional information of the target device; and
generating the target model of the target device based on the three-dimensional information.

6. The system of claim 5, wherein the three-dimensional information of the target device is acquired by at least one of a structured-light camera, a binocular camera, or a distance measuring device.

7. The system of claim 4, wherein the generating the target model of the target device includes:
obtaining critical posture information of the operating apparatus at one or more critical statuses;
determining, based on the critical posture information, critical model information of one or more critical models of the target device each of which corresponds to one of the one or more critical statuses of the operating apparatus; and
generating the target model of the target device based on the critical model information of the one or more critical models of the target device.

8. The system of claim 7, wherein the critical model information includes dimension information of the one or more critical models.

9. The system of claim 7, wherein the determining, based on the critical posture information, critical model information of one or more critical models of the target device each of which corresponds to one of the one or more critical statuses of the operating apparatus includes:
for each of the one or more critical statuses of the operating apparatus,
determining, based on critical posture information of the operating apparatus at the critical status, a growth origin and a growth direction of a critical model that corresponds to the critical status; and
determining the critical model information of the critical model according to the growth origin and the growth direction.

10. The system of claim 7, wherein the generating the target model of the target device based on the critical model information of the one or more critical models of the target device includes:
determining target model information by combining the critical model information of the one or more critical models; and
generating the target model of the target device based on the target model information.

11. A system, comprising:
at least one storage medium including a set of instructions; and
at least one processor configured to communicate with the at least one storage medium, wherein when executing the set of instructions, the at least one processor is configured to direct the system to perform operations including:
obtaining critical posture information of an operating apparatus at one or more critical statuses;
determining, based on the critical posture information, critical model information of one or more critical models of a target device each of which corresponds to one of the one or more critical statuses of the operating apparatus;
generating a target model of the target device based on the critical model information of the one or more critical models of the target device;
determining first posture information of the target model and second posture information of the operating apparatus; and
generating a collision prediction between the target model and the operating apparatus based on the first posture information and the second posture information, the collision prediction indicating whether there is a space accommodating the target device during an operation of the operating apparatus.

12. A method implemented on a computing device having a processor and a computer-readable storage device, the method comprising:
obtaining a target model of a target device;
determining first posture information of the target model and second posture information of an operating apparatus, wherein the target device is set on an end of the operating apparatus; and
generating a collision prediction between the target model and the operating apparatus based on the first posture information and the second posture information, the collision prediction indicating whether there is a space accommodating the target device during an operation of the operating apparatus.

13. The method of claim 12, wherein the operating apparatus includes a mechanical arm, and the target device is set on an end of the mechanical arm.

14. The method of claim 12, wherein the obtaining a target model of a target device includes:
identifying, according to a target identification of the target device, the target model of the target device from a plurality of device models stored in a storage device; and
retrieving the target model from the storage device.

15. The method of claim 12, wherein the obtaining a target model of a target device includes:
generating the target model of the target device in response to a model generation operation.

16. The method of claim 15, wherein the generating the target model of the target device includes:
obtaining three-dimensional information of the target device; and
generating the target model of the target device based on the three-dimensional information.

17. The method of claim 16, wherein the three-dimensional information of the target device is acquired by at least one of a structured-light camera, a binocular camera, or a distance measuring device.

18. The method of claim 15, wherein the generating the target model of the target device includes:
obtaining critical posture information of the operating apparatus at one or more critical statuses;
determining, based on the critical posture information, critical model information of one or more critical models of the target device each of which corresponds to one of the one or more critical statuses of the operating apparatus; and
generating the target model of the target device based on the critical model information of the one or more critical models of the target device.

19. (canceled)

20. The method of claim 18, wherein the determining, based on the critical posture information, critical model information of one or more critical models of the target device each of which corresponds to one of the one or more critical statuses of the operating apparatus includes:
for each of the one or more critical statuses of the operating apparatus,
determining, based on critical posture information of the operating apparatus at the critical status, a growth origin and a growth direction of a critical model that corresponds to the critical status; and
determining the critical model information of the critical model according to the growth origin and the growth direction.

21. The method of claim 18, wherein the generating the target model of the target device based on the critical model information of the one or more critical models of the target device includes:
determining target model information by combining the critical model information of the one or more critical models; and
generating the target model of the target device based on the target model information.

22-24. (canceled)
